(19) United States
(12) Patent Application Publication
(10) Pub. No.: US 2005/0079213 A1
(43) Pub. Date: Apr. 14, 2005

(54) Pharmaceutical compositions

(75) Inventors: Ma. Teresa Y. Tan, Quezon City (PH); Eulogio Singh, Rizal (PH); Rita Josefina M. Santos, Quezon City (PH); Kennie U. Dee, Quezon City (PH)

Correspondence Address: BROWN & MICHAELS, PC;400 M & T BANK BUILDING, 118 NORTH TIOGA ST, ITHACA, NY, 14850, US

(73) Assignee: Unilab PharmaTech Ltd, Central Hong Kong (CN)

(21) Appl. No.: 10/826,098

(22) Filed: Apr. 16, 2004

(30) Foreign Application Priority Data
Jun. 6, 2003 (PH) 12003000285

(51) Int. Cl. A61K 31/545; A61K 9/48; A61K 9/20

(52) U.S. Cl. 424/451; 514/200

(57) ABSTRACT

A solid oral dosage composition of cefuroxime axetil comprising a tablet inside a capsule, the capsule serving to mask the bitter taste of the drug upon oral administration. This tablet-in-a-capsule format is bioequivalent to the commercial film-coated tablet.

### REFERENCE TO RELATED APPLICATIONS

This application claims an invention which was disclosed in Republic of the Philippines Patent Application No. 12003000285, filed Jun. 6, 2003, entitled “PHARMACEUTICAL COMPOSITIONS”. Pursuant to 35 U.S.C. § 119 (a)-(d) and (f), 35 U.S.C. § 172 and 35 U.S.C. § 365(a) and (b), the benefit of the earlier-filed foreign application is hereby claimed, and the aforementioned application is hereby incorporated herein by reference in its entirety.

### BACKGROUND OF THE INVENTION

1. Field of the Invention

The present invention relates to a solid oral dosage composition of cefuroxime axetil comprising a tablet inside a capsule, the capsule serving to mask the bitter taste of the drug upon oral administration. It has been found that this tablet-in-a-capsule format is bioequivalent to the commercial film-coated tablet.

2. Description of Related Art

Cefuroxime, as disclosed in U.S. Pat. No. 3,974,153, is a broad spectrum second-generation cephalosporin characterized by high activity against a wide range of gram-positive and gram-negative bacteria, this property being enhanced by the very high stability of the compound to B-lactamases produced by a range of gram-negative microorganisms. Cefuroxime and its salts are used as injectable antibiotics since they are poorly absorbed from the gastro-intestinal tract.

Esterification of the carbonyl group of cefuroxime as a 1-acetoxyethyl ester to give cefuroxime axetil improves the effectiveness on oral administration as described in U.S. Pat. No. 4,562,181. This patent further discloses that it is particularly advantageous to use cefuroxime axetil in its amorphous form to enhance dissolution and hence bioavailability.

Cefuroxime axetil has an extremely bitter taste which is long lasting and which cannot be adequately masked by addition of sweeteners and flavors. The tablet needs to be film-coated to eliminate the bitter taste. However, as described in U.S. Pat. No. 4,897,270, the film coating must rupture in less than 40 seconds when measured by a rupture test wherein the tablet is placed in a beaker of still 0.07 N hydrochloric acid at 37° C. When the film coating is too thick, the slow permeation of water through the film coating to the core will cause gel formation of the amorphous cefuroxime axetil core leading to poor dissolution and bioavailability. The rupture time of less than 40 seconds for the film coating prevents gel formation while at the same time providing adequate barrier against the bitter taste of the medicine. Amorphous cefuroxime axetil film-coated tablets are commercially available from Glaxo USA under the brand name Ceftin®.

### SUMMARY OF THE INVENTION

The present invention provides a solid oral dosage composition of cefuroxime axetil comprising a tablet inside a capsule, the capsule serving to mask the bitter taste of the drug upon oral administration. It has been found that this tablet-in-a-capsule format is bioequivalent to the commercial film-coated tablet.

### DETAILED DESCRIPTION OF THE INVENTION

The present invention comprises a core tablet of substantially amorphous cefuroxime axetil inside a capsule. The core tablet is preferably shaped like a capsule (caplet).

The core tablet comprises more than 10% w/w of a distintegrant, preferably more than 15% w/w, and most preferably 20% w/w. The disintegrant includes but is not limited to starches, clays, celluloses, algins, gums, cross-linked polymers, and combinations thereof. The preferred disintegrants are microcrystalline cellulose, starch, croscarmellose, crospovidone, sodium starch glycolate, and combinations thereof.

In addition to the active ingredient and disintegrant(s), the core tablet may contain a number of other ingredients referred to as excipients. These excipients include among others diluents, binders, lubricants, glidants, and colorants.

The core tablet is filled into a capsule which is generally a two-piece hard gelatin capsule, but capsules made from hydroxypropylmethylcellulose, vegetable or plant-based cellulose, polysaccharides and other polymers can also be used.

We have surprisingly found that the composition of this instant invention is bioequivalent to the commercial film-coated tablet even if the rupture time of the capsule is in excess of 60 seconds. In contrast, the same amount of formulation filled into capsules without tabletting results in gel formation and consequently poor dissolution. Not wishing to be bound by theory, it is believed that tabletting results in a higher disintegration force that causes the rupture of the capsule by the caplet before gel formation occurs, especially in the central overlap region of the capsule which is twice as thick as ends.

The thickness and width of the caplet is preferably greater or equal to 65% of the internal diameter of the capsule, more preferably greater or equal to 75%, and most preferably greater or equal to 80%.

### EXAMPLE 1

| Ingredients | Mg/capsule |
|---|---|
| Amorphous Cefuroxime Axetil | 301.6* |
| Starch | 93.6 |
| Croscarmellose sodium | 66.0 |
| Sodium lauryl sulfate | 5.0 |
| Colloidal silicon dioxide | 1.5 |
| Total weight | 467.7 |

*Equivalent to 250 mg of cefuroxime

Cefuroxime axetil, starch, croscarmellose, sodium lauryl sulfate, and colloidal silicon dioxide were blended together, and compacted into granules with a roller compactor. The granules were filled into size no. 1 two-piece hard gelatin capsule.

Dissolution was carried out according to USP 26 in 900 ml of 0.07 N HCl at 37° C., in USP apparatus II.

| | Cumulative percent |
|---|---|
| Time (min) | drug released |
| 15 | 52.4% |
| 45 | 65.7% |

The dissolution fails to comply with the USP requirement for cefuroxime axetil of not less than 65% dissolved in 15 minutes, and not less than 75% in 45 minutes. Gel formation was observed in the central overlap region of the capsule; this gel persisted even after the capsule has dissolved.

### EXAMPLE 2

The granules of Example 1 were compressed into 467.7 mg caplets using a Manesty BB3B tabletting machine. The size of the caplet is 18 mm×5.7 mm×5.1 mm (length×width×thickness) with a hardness of 6-10 kp. The caplets were manually filled into size no. 1 two-piece hard gelatin capsules. The dimension of the capsule is 19.4 mm×6.4 mm (length×internal diameter).

Dissolution was carried out according to USP 26 in 900 ml of 0.07 N HCl at 37° C., in USP apparatus II.

| | Cumulative percent |
|---|---|
| Time (min) | drug released |
| 15 | 92.6% |
| 45 | 98.5% |

The dissolution complies with the USP requirement for cefuroxime axetil of not less than 65% dissolved in 15 minutes, and not less than 75% in 45 minutes. Gel formation was not observed. The mean rupture time of the caplet-in-capsule is about three minutes.

Comparison of Example 1 and Example 2 clearly shows suppression of gel formation and thus improved dissolution when the same amount of granules is tabletted before filling into the capsule.

### EXAMPLE 3

| Ingredients | Mg/capsule |
|---|---|
| Amorphous Cefuroxime Axetil | 603.2* |
| Starch | 187.1 |
| Croscarmellose sodium | 32.1 |
| Sodium lauryl sulfate | 10.0 |
| Colloidal silicon dioxide | 3.0 |
| Total Weight | 835.4 |

*Equivalent to 500 mg of cefuroxime

Cefuroxime axetil, starch, croscarmellose, sodium lauryl sulfate, and colloidal silicon dioxide were blended together, and compacted into granules with a roller compactor. The granules were filled into size no. 00 two-piece hard gelatin capsule.

Dissolution was carried out according to USP 26 in 900 ml of 0.07 N HCl at 37° C., in USP apparatus II.

| | Cumulative percent |
|---|---|
| Time (min) | drug released |
| 15 | 35.4% |
| 45 | 42.2% |

The dissolution fails to comply with the USP requirement for cefuroxime axetil of not less than 65% dissolved in 15 minutes, and not less than 75% in 45 minutes. Gel formation was observed in the central overlap region of the capsule; this gel persisted even after the capsule has dissolved.

### EXAMPLE 4

The granules of Experiment 3 were compressed into 835.4 mg caplets using a Kilian tablet press. The size of the caplet is 20 mm×6.0 mm×7.2 mm (length×width×thickness) with a hardness of 7-11 kp. The caplets were manually filled into size no. 00 two-piece hard gelatin capsules. The dimension of the gelatin capsule is 23.3 mm×7.9 mm (length×internal diameter).

Dissolution was carried out according to USP 26 in 900 ml of 0.07 N HCl at 37° C., in USP apparatus II.

| | Cumulative percent |
|---|---|
| Time (min) | drug released |
| 15 | 96.6% |
| 45 | 100% |

The dissolution complies with the USP requirement for cefuroxime axetil of not less than 65% dissolved in 15 minutes, and not less than 75% in 45 minutes. Gel formation was not observed. The mean rupture time of the caplet-in-capsule is about three minutes.

Comparison of Example 3 and Example 4 clearly shows suppression of gel formation and thus improved dissolution when the same amount of granules is tabletted before filling into the capsule.

The bioavailability of the caplet-in-capsule formulation of Example 4 was compared to Glaxo's 500 mg film-coated tablet (Ceftin®).

COMPARISON OF PHARMACOKINETIC PARAMETERS

| Parameter | Example 4 | Ceftin ® |
|---|---|---|
| Tₘₐₓ ± S.D. | 2.0 ± 0.68 h | 2.0 ± 0.74 h |
| Cₘₐₓ ± S.D. | 5.48 ± 1.53 mcg/ml | 5.05 ± 1.58 mcg/ml |
| % reference | 108% | reference |
| AUC_{0-12 h} ± S.D. | 22.94 ± 3.32 mcg/ml-h | 19.74 ± 5.17 mcg/ml-h |
| % reference | 116% | reference |

The bioavailability study was carried out in 18 volunteers under fasting conditions using a single oral dose equivalent to 500 mg of cefuroxime. The above data shows that the caplet-in-capsule formulation of Example 4 is bioequivalent to the commercial film-coated Ceftin® tablet.

While the present invention has been described in terms of its specific embodiments, certain modifications and equivalents will be apparent to those skilled in the art and are intended to be included within the scope of the present invention.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A pharmaceutical composition for oral administration which comprises a core tablet of cefuroxime axetil inside a capsule.

2. The composition according to claim 1, wherein the tablet is in the shape of a capsule (caplet).

3. The composition according to claim 2, wherein the smallest dimension of the caplet is greater or equal to 65% of the internal diameter of the capsule.

4. The composition according to claim 3, wherein the smallest dimension of the caplet is greater or equal to 75% of the internal diameter of the capsule.

5. The composition according to claim 4, wherein the smallest dimension of the caplet is greater or equal to 80% of the internal diameter of the capsule.

6. The composition according to claim 1, wherein the core tablet contains more than 10% w/w of a disintegrant.

7. The composition according to claim 6, wherein the core tablet contains more than 15% w/w of a disintegrant.

8. The composition according to claim 7, wherein the core tablet contains more than 20% w/w of a disintegrant.

9. The composition according to claims 6, 7, and 8, wherein the disintegrant is selected from microcrystalline cellulose, starch, croscarmellose, crospovidone, sodium starch glycolate, and combinations thereof.

10. The composition according to claim 1, wherein the cefuroxime axetil is substantially amorphous.

11. The composition according to claim 1, wherein the capsule is a two-piece hard gelatin capsule.

12. The composition according to claim 1, wherein the capsule is a two-piece hydroxypropylmethylcellulose capsule.

13. The composition according to claim 1, wherein the capsule is a two-piece capsule made of vegetable or plant-based cellulose.

14. The composition according to claim 1, wherein the capsule is a two-piece capsule made of polysaccharide.
